(19) United States
(12) Patent Application Publication
(10) Pub. No.: US 2003/0185915 A1
(43) Pub. Date: Oct. 2, 2003

(54) Synergetic composition for the treatment of psoriasis and other skin disorders and method therefor

(76) Inventors: Jaime Carlo, Tampa, FL (US); Nestor Chinea, Bayamon, PR (US); Carmelo Rivera, Yauco, PR (US); Juan Franco, San Juan, PR (US)

Correspondence Address: EUGENIO J. TORRES, FERRAMAR BUILDING, SUITE 1, 1060 ASHFORD AVENUE, SAN JUAN, PR, 00907, US

(21) Appl. No.: 10/109,453

(22) Filed: Mar. 28, 2002

(51) Int. Cl. A61K 35/78

(52) U.S. Cl. 424/744

(57) ABSTRACT

Synergetic compounded medication formula for the treatment of psoriasis, seborrhea, dermatitis, dandruff, eczema, acne, and other skin disorders. The present invention is to provide regenerative treatment of skin disorders recurrent in all areas of the body. The invention of this disclosure uses a well-known corticosteroid as an active ingredient, namely Triamcinolone acetonide, which when used in combination with a special formula is effective, easy to use, and less expensive than similar products available with a prescription in the market. A method for administering said composition to inhibit proliferation of psoriatic cell populations in the epidermis is disclosed

### STATEMENT REGARDING FEDERALLY SPONSORED RESEARCH AND DEVELOPMENT

N/A

### RELATED APPLICATIONS

N/A

### BACKGROUND OF THE INVENTION

1. Field of the Invention

The present invention relates generally to a composition and method for the topical treatment of psoriasis and other skin disorders and, more particularly, to a composition for the treatment of psoriasis comprising therapeutically effective doses of zinc pyrithione, triamcinolone acetonide, and polysorbate 80, incorporated into at least one suitable topical medication carrier, and combined, optionally, with anti-oxidants, anti-fungal, and/or anti-bacterial agents.

2. Discussion of the Background

1. Psoriasis

Psoriasis is a condition in which cell proliferation is increased up to 10 times the normal rate for an individual. The skin is the largest portion of the human body which is comprised of cells within three skin layers. Each of the skin layers is in a constant state of growth with the outer layer being formed of predominantly dead tissue which is naturally being discarded at a normal rate. Replacement of cells from underlying layers is accomplished by cell division and maturation where cells move upwardly and outwardly at a rate which varies dependent upon the age, sex, and/or health of an individual. Psoriasis causes an increased turn over of cells, which in turn increases the rate of cell growth and cell death. This increased rate of cell growth and cell death may result in injuries and/or disorders which accompany the increased synthesis of all tissue components and further elevate the strain placed upon skin or other tissue and the bio-synthetic capabilities of the cells within the affected area.

The word “psoriasis” comes from ancient Greece and means “to itch.” Psoriasis is a chronic skin disease long recognized for its peculiar clinical symptoms characterized by circumscribed red patches covered with white scales, and often accompanied by varying degrees of discomfort. It has been estimated that psoriasis affects about 2 percent of the population in Western countries, 0.1 to 0.3 percent in the Far East and is rather rare in persons of the black race. Thus, psoriasis is one of the most common dermatologic diseases which, according to National Psoriasis Foundation's (“NPF's”) estimates, affects more than 6.4 million people in the U.S. to some degree.

Generally, psoriasis appears as patches of raised red skin that vary in size between one to several centimeters. These eruptions appear on the surface of the skin and begin to itch. These areas form plaques over the reddened lesions. The plaques resemble multi-layered scales of skin. In the area where scales have been shed, tiny bleeding points called “Auspitz sign” appear.

The major pathophysiological events involved in the disease process are accelerated epidermal proliferation and metabolic activity, proliferation of capillaries in the dermal region, and invasion of the dermis and epidermis by inflammatory cells. At the cellular level, psoriasis is a benign proliferative disease of keratinocytes of unknown etiology. Although the disease appears to be inherited, its mode of transmission is not known. Furthermore, the disease can be triggered or exacerbated by external factors such as trauma, infection and drugs.

Histologically, the skin pathology is characterized by acanthosis, thickening of the epidermis, angiogenesis of superficial blood vessels and an inflammatory response. It is not known whether the primary alteration in psoriasis resides in the keratinocytes or is the result of an autoimmune process. It is also known that psoriatic zones lose water 8-10 times faster than normal healthy skin. The areas of skin affected by psoriasis therefore tend to have increased metabolic rates, which, in turn, has a negative impact on tissue catabolism and potentially causes muscle wasting.

Psoriasis most commonly appears on the scalp, the knees, elbows, and torso. But psoriasis can develop anywhere, including the nails, palms, soles, genitals, and face (which is rare). Often the lesions appear in a symmetrical fashion, in the same place on the right and left sides of the body. When a person suffers from psoriasis, red maculae, or red papules having clear borders, occur on portions of the patient's body, which are susceptible to external phlogogenic (inflammatory) stimuli, such as the head, elbows, knees, and buttocks, and on areas where bacteria and fungi are likely to proliferate, such as pilose (hairy) regions of the body.

Psoriasis appears to be slightly more prevalent in women than men. The average age of diagnosis is 28, and psoriasis most commonly appears between the ages of 15 and 35. However, it can develop at any time—a first-time diagnosis of psoriasis has been seen in very old people and in newborn babies and small children. Psoriasis in infants is considered rare, although between 10 percent and 15 percent of those with psoriasis get it before age 10. About 150,000 to 260,000 new cases of psoriasis are diagnosed each year.

No special blood test or other diagnostic tool exists for psoriasis. The diagnosis is usually determined through examination of the skin by a physician or other health care provider. Less commonly, a skin biopsy is examined under the microscope for biological evidence of psoriasis. The presence of small pits in the fingernails is also an indicator of psoriasis.

Psoriasis comes in different forms and varies in intensity from a few random spots to a massive outbreak covering the entire body and requiring hospitalization, but the underlying problem in all types of psoriasis is overproduction of epidermal cells. Instead of adhering to the 21 to 28 day cycle of cell turnover, those afflicted with psoriasis race through the cycle in 3 or 4 days. The epidermis may grow to 5 to 10 times its normal thickness. The thickened epidermis, overgrowth of blood vessels, and infiltrate of neutrophils and lymphocytes account for the psoriatic lesions being raised and easily palpable.

The etiology of psoriasis is still poorly understood, however evidence has accumulated clearly indicating a role for T cells. Hordes of activated T cells are found in psoriatic skin and almost none in healthy skin. It has also been discovered that these activated T cells secrete interleukin-6, which has as one of its effects the ability to stimulate skin cell growth. The infiltration of activated white blood cells suggests that an immune response has been mustered against something.

There are various theories about what that “something” is. One suggests a genetic abnormality. Other theories suggest that psoriasis is an immune system overreaction against invading organisms, or toxins produced by those organisms (“superantigens”), as it is known that latent psoriasis can be activated by various infections as well as certain drugs and injury to the skin. Bone marrow transplantation has resulted in clearance of the disease. In any event, psoriasis is currently considered to be an immunologic skin disorder. In psoriasis, the immune system is somehow triggered, which in turn speeds up the growth cycle of skin cells, which pile up on the surface and form the elevated red lesions when the body can't shed them fast enough.

Psoriasis is not contagious. It is not something one “catch” or “pass on.” Lesions of psoriasis may be unsightly, but they should not be regarded as an infection or an open wound. Thus, an individual with psoriasis poses no threat to the health or safety of others.

Psoriasis is considered to be a pluricausal hereditary disease whose onset occurs due to the genetic makeup in the body. Since it is known that psoriasis has a close relationship with histocompatibility antigen (HLA) which exhibits polymorphism due to the variation of the HLA gene, it is clear that psoriasis is a hereditary disease. Psoriasis has a genetic component that makes certain people more likely to develop it. A family association exists in one out of three cases. However, it is not possible to predict who will get psoriasis. Environmental factors may trigger the onset of psoriasis, even in people without an apparent family history of psoriasis. These triggers, which can cause the body to go from a very mild case to a severe case within days, may include emotional stress, injury to the skin, some types of infections and reaction to certain drugs, climates, or foods.

Once psoriasis begins, there are only remissions and relapses of varying degrees of intensity. There is no known cure, only possible control over the severity, but there are many different treatments, both topical and systemic, that can control the symptoms (itching, flaking, and red patches) for periods of time. Experimentation is often required to find a treatment that works for a particular person. Some people who have psoriasis experience spontaneous remissions, but no one knows why this happens and they are unpredictable. Once a psoriasis outbreak occurs, the body will continue to utilize the nails, scalp, and skin to achieve balance. The psoriasis sufferer can achieve control and may even be spot free for years, but that is not considered “cured”.

Although psoriasis is rarely life-threatening, about 400 people die from complications caused by psoriasis each year. Primarily, such complications occur in relation to a severe, extensive form of psoriasis, such as generalized pustular psoriasis or erythrodermic psoriasis, where large areas of skin are shed. The skin plays an important role in regulating body temperature and serving as a barrier to infection. When a person's skin is compromised to such a great extent, secondary infections are possible. Fluid loss is a complicating factor in these serious forms of psoriasis, and a great strain is also placed on the circulatory system. Also, approximately 400 people receive social security disability due to psoriasis each year.

Psoriasis is a difficult and expensive disease to treat. A typical patient may spend hundreds or even thousands of dollars a year in medications and other modalities of treatment just to find a relief of their symptoms. This does not take into account the number of hours each month patients have to spend treating their disease, visiting their physician, or traveling to a phototherapy center. Psoriasis is a chronic illness that, in many cases, requires continuous treatment. The cost of medications and visits to the doctor are ongoing. Severe cases may require periods of hospitalization. Most therapy costs are covered by insurance, but it can be quite expensive. It is estimated that 56 million hours of work are lost each year by people who suffer from psoriasis and between $1.6 billion and $3.2 billion is spent annually on treating psoriasis.

Psoriasis has a physical impact on the skin, but it also affects people's feelings, behaviors, and experiences. Due to the characteristic formation of skin lesions and eruptions, psoriasis gives its victims an unfavorable psychological outlook on life. Recognizing and acknowledging the social implications of psoriasis is an important step toward learning to cope with the disease. Psoriasis can be a mildly annoying problem or can destroy the self-esteem and life of the victim. Although it is not contagious, it is an ugly disease that can alienate coworkers and acquaintances. For the most part, people with psoriasis function normally. However, psoriasis is often misunderstood by the public, which can make social interactions awkward. This may lead to emotional problems such as anxiety, anger, embarrassment and depression. In addition, psoriasis can affect the type of work people do if it is visible.

Psoriasis marks people as different, because the skin—that which “displays” the human being inside—has an altered appearance. The public may react with great insensitivity and ignorance at unsightly patches of psoriasis, because they don't know what it is and it is something they are not used to seeing. People may not understand why they developed psoriasis, or even what the disease really is. There is no cure and it can take lots of experimentation before an effective, tolerable treatment is found. Therefore, having psoriasis can generate great feelings of frustration.

2. Types of Psoriasis

There are several types of psoriasis with different symptoms and degrees of severity. Various symptoms and phenomena are observed in all types of psoriasis, such as hyperplasia and abnormal cornification of epidermal cells ascribed to the excess turnover of the cells by hypermetabolism.

2.1 Plaque Psoriasis

Plaque psoriasis is the most typical form of the disease-four out of five people with psoriasis have this type. A “plaque,” is the name used to describe the well-defined patches of red, raised skin, and the word “lesion” is also commonly used. The technical name for plaque psoriasis is psoriasis vulgaris (vulgaris means common). The flaky, silvery white buildup on top of the plaques is called scale; it is composed of dead skin cells. This scale comes loose and sheds constantly from the plaques. Skin affected with psoriasis is generally very dry, and other possible symptoms include skin pain, itching, and cracking. Plaque psoriasis can appear on any skin surface, although the knees, elbows, scalp, trunk, and nails are the most common locations.

2.2 Guttate Psoriasis

Guttate psoriasis is characterized by small dot-like lesions and resembles small, red, individual drops on the skin (the word guttate comes from the Latin word meaning “drop”). These lesions generally appear on the trunk and limbs, and sometimes on the scalp, and they are usually not as thick or as scale-covered as plaque psoriasis. Guttate psoriasis often starts in childhood or young adulthood, and it may be triggered by an infection of some sort.

2.3 Inverse Psoriasis

Inverse psoriasis, also called flexural psoriasis, is characterized by intense inflammation and little scaling and is found in the armpits, groin, under the breasts and in other skin folds around the genitals and buttocks. This type of the disease appears as smooth, dry areas of skin that are red and inflamed but do not have the scaling associated with plaque psoriasis. Inverse psoriasis is particularly subject to irritation from rubbing and sweating because of its location in skin folds and tender areas. It is more common and troublesome in overweight people.

2.4 Erythrodermic Psoriasis

Erythrodermic psoriasis is a particularly inflammatory form of psoriasis characterized by intense sloughing and inflammation of the skin and that often affects most of the body surface. The erythma (reddening) and exfoliation (shedding) of the skin are often accompanied by severe itching and pain. Swelling may also develop. It is the least common form of the disease. It most commonly appears on people who have unstable plaque psoriasis, where lesions are not clearly defined.

2.5 Generalized Pustular Psoriasis

Pustular psoriasis is characterized by weeping lesions and intense scaling. Pustular psoriasis spread over wide areas of the body is also called von Zumbusch pustular psoriasis, named after the physician who first described it in the early 1900s. In this relatively rare form of the disease, widespread areas of reddened skin (erythema) develop, and the skin becomes acutely painful and tender. Pustules—blisters of non-infectious pus—may appear on the skin, dry, and then reappear in repeated cycles lasting several days.

2.6 Localized Pustular Psoriasis

Pustules of psoriasis can be confined to local areas, particularly the hands and feet. The form called palmo-plantar pustulosis (PPP) is characterized by large (up to 0.5 cm, or about the size of a pencil eraser) pustules in fleshy areas of hands and feet, such as the base of the thumb and the sides of the heels. The pustules appear in a studded pattern throughout reddened plaques of skin, then turn brown and peel. Another rare form is called acropustulosis (or acrodermatitis continua of Hallopeau). In this type, skin lesions develop on the ends of the fingers and sometimes the toes. The lesions can be painful and disabling, with nail deformities and, in severe cases, changes to the bone.

3. Psoriatic Arthritis (“PA”)

PA is a specific type of arthritis that develops in approximately 20 percent of people who have psoriasis. This disease is present in up to 0.1% of world population and it usually begins between 30-50 years of age, but it may develop at any time, and affects men and women equally.

Joints commonly affected by PA are the wrists, knees, ankles, lower back and neck. But PA primarily involves the distal interphalangeal joints of fingers or toes. Asymmetric involvement of large and small joints, as well as of sacroiliac joints and spine, is common. PA can at times be quite destructive, progressing to chronic arthritis and arthritis mutilans, with extensive destruction of large and small joints. The clinical course of PA is long term, with characteristic remissions and relapses. In most cases, psoriasis may precede the onset of PA, with a very variable latency time (up to 10 years), however there are also forms in which PA may precede the onset of psoriasis.

The exact cause of the disease is unknown, although interplay of immune, genetic, and environmental factors are suspected. This joint disease can be difficult to diagnose, particularly in its milder forms. PA can start slowly with mild symptoms, or it can develop quickly. Left untreated, PA can be a progressively disabling disease.

A research study released by the NPF on Jan. 16, 2002, showed that approximately 1 million U.S. adults have PA, a number significantly higher, roughly double, than previously believed by the medical community. In addition, the research also suggested that many people with psoriasis may have PA and not know it. The survey showed a strong link between PA and psoriasis. About 85 percent of those with PA reported in the survey that they also had psoriasis.

PA symptoms include stiffness, pain, swelling and tenderness of the joints and the soft tissue around them, reduced range of motion, morning stiffness and tiredness, nail changes, including pitting or lifting of the nail-found in 80 percent of people with PA-, and redness and pain of the eyes, such as conjunctivitis.

3.1 Types of PA

3.1.1 Symmetric Arthritis

This form of PA is much like rheumatoid arthritis, but generally milder and with less deformity. It usually affects multiple symmetric pairs of joints and can be disabling. The associated psoriasis is often severe. About 50 percent of people with this form of PA will develop varying degrees of progressive, destructive disease, which can be disabling.

3.1.2 Asymmetric Arthritis

Asymmetric (not occurring in the same joints on both sides of the body) arthritis usually involves only one to three joints. It can affect any joint, such as the knee, hip, ankle or wrist. It could involve just one finger or a number of them. The hands and feet have enlarged “sausage” digits, caused by swelling and inflammation of tendons. The joints may be warm, tender, and red. Individuals may experience periodic joint pain that is usually responsive to medical therapy. This form is generally mild, although some people will develop disabling disease.

3.1.3 Distal Interphalangeal Predominant (DIP)

This form of arthritis, although the “classic” type, occurs in only about 5 percent of people with PA. Primarily, it involves the distal joints of the fingers and toes (the joint closest to the nail). Sometimes it is confused with osteoarthritis, but nail changes are usually prominent.

3.1.4 Spondylitis

In about 5 percent of individuals, inflammation of the spinal column is the predominant symptom. Inflammation with stiffness of the neck, lower back, sacroiliac or spinal vertebrae are common symptoms in a larger number of patients, making motion painful and difficult. Peripheral disease can be present in the hands, arms, hips, legs and feet. Spondylitis, when severe, may be associated with generalized symptoms.

3.1.5 Arthritis Mutilans

This is a severe, deforming, and destructive arthritis that affects fewer than 5 percent of people with PA. It principally affects the small joints of the hands and feet, though there is frequently associated neck or lower back pain. This type can progress over months and years. Arthritic flares and remissions tend to coincide with skin flares and remissions.

4. Measuring the Severity of Psoriasis

Psoriasis is divided into three degrees of severity: mild, moderate, and severe. About 75 percent to 80 percent of people with psoriasis have what is considered mild disease, and about 20 percent to 25 percent have moderate to severe psoriasis. These categories are useful for selecting what treatments might be appropriate for individuals with psoriasis. The physical measure of severity is based on how much skin on the body is affected by psoriasis. As a general rule of thumb, the palm of the hand represents 1 percent of the body's surface. Severity can also hinge on how psoriasis affects a person's quality of life. If psoriasis covers only a small area, yet is serious enough to be disabling—for example, bad psoriasis of the hands or feet—, it could be considered a severe case of the disease.

4.1 Mild Psoriasis

People with psoriasis on less than 2 percent of their body are considered to have a mild case. Generally, isolated patches of psoriasis are found on the knees, elbows, scalp, and hands and feet. Topical treatments—including moisturizers and over-the-counter (“OTC”) and prescription creams, ointments, and shampoos—are usually sufficient to keep the psoriasis in check

4.2 Moderate Psoriasis

Moderate psoriasis is defined as affecting between 2 and 10 percent of the body's surface. Psoriasis may appear on the arms, the legs, torso, scalp, and other areas. Appropriate therapies include topical treatments, phototherapy, and oral medications depending on the location and extent of the psoriasis and other individual factors.

4.3 Severe Psoriasis

Psoriasis covering more than 10 percent of the body is considered severe. Extensive areas of skin may be covered with psoriasis plaques or pustules, or widespread erythrodermic psoriasis can cause severe peeling of the skin. People with severe psoriasis are more likely to develop psoriatic arthritis. Powerful treatments, including phototherapy, oral medications, or a combination of these, are usually necessary to manage severe psoriasis.

5. Psoriasis Therapies

In the past, the treatment of psoriasis has included the use of various chemical agents and the use of ultraviolet (“UV”) light. Alternatively, therapeutic regimes for the treatment of psoriasis include topical administration of corticosteroids, anthralin, or coal tar preparations, among others, on the affected areas. Treatments for psoriasis can be divided into three basic categories: sunlight and topical agents (external therapies); phototherapy (artificial UV light, or a combination of UV light and medications); and, systemic (internal) medications taken by pill or injection.

Sunlight and topical treatments are the weakest treatments that also usually have the fewest side effects. These treatments are mostly used for mild psoriasis, and stronger therapies may become necessary as psoriasis becomes more severe. Nevertheless, all of these methods have generally not provided satisfactory treatment of psoriasis for individuals. In addition, all of them involve remarkable side effects, which can induce discontinuation of the treatment

As is known in the art, no single therapy is ideal for the treatment of psoriasis and it is extremely rare for a patient to not receive treatment from several different therapeutic alternatives to attempt to prevent relapse and/or remission of the disease. In addition, individuals are frequently required to be exposed to increased doses of medication, which may magnify side effects adversely affecting the health of the individual.

Treating psoriasis can be a challenging and long-term proposition, because the disease is chronic and unpredictable. Even though there is no cure for psoriasis or PA, people have a variety of treatment options at their disposal to help them gain control over the symptoms associated with these diseases. The selection of therapy may be based, among other things, on the location of the lesions, the age of the patient, the type of psoriasis, its severity, and the presence of other illnesses. Some experimentation may be required before an effective approach is discovered. The goal in selecting a therapy is to find an approach that has the best results and the fewest side effects. It is generally recommended with prior art treatments that people with psoriasis start with the least potent treatments and move to stronger ones until they find an acceptable combination of results and risks. A physician, particularly a dermatologist, can provide guidance in selecting the right therapy.

5.1 Topical Therapy

Topical therapy is the first-line approach. Several topical treatments are available, such as corticosteroids, coal tar, anthralin, calcipotriene, and tazarotene.

5.1.1 Steroids

Topical corticosteroids remain the mainstay of psoriasis therapy since first introduced in the 1950s. This class of topical medication is one of the most commonly prescribed therapies for mild to moderate psoriasis. Steroids are synthetic drugs made to resemble hormones that occur naturally in the body, such as cortisone. Topical corticosteroids are available in many strengths and vehicle formulations such as lotions, creams, emollients, ointments, gels, sprays, and foams. Their relative potencies have been ranked in the Stoughton-Cornell classification based on their vasoconstrictive capacity ranging from one to seven in a descending potency scale. Hydrocortisone cream 1%, available without prescription, is the prototype of a class 7 topical corticosteroid. At the other end of the scale, class 1, also known as superpotent corticosteroids, are, among others, Clobetazole proionate, Alobetazole propionate, Betamethazone dipropionate augmented, and Diflorozone diacetate. An extensive number of other steroids fill in the other categories. It must be noted that the same steroid preparation is considered more potent when based in an ointment vehicle versus a cream preparation. Even though steroids come in a wide range of strengths, the weakest OTC steroids are not helpful in treating psoriasis. Stronger steroids are usually more effective, but the risk of side effects is greater, and some potent steroids are not appropriate for psoriasis in certain areas (such as the skin folds or genitals).

The external or topical use of a steroid has the immediate effect of reducing the symptoms of psoriasis, particularly the reduction of eruptions. However, the administration of the adrenocortical hormone over the long periods of time that are necessary in such treatment causes tachyphylaxis, that is, an increased resistance and tolerance buildup, so that the dose must be increased, or stronger drugs must be used in order to obtain the desired therapeutic effect. Occasionally, the occurrence of a new lesion is observed at a site which has been treated with the drug. In addition, when adrenocortical hormone is applied to skin in the form of a coating, ointment, salve or paint, the hormone exerts its action not only on the lesion but also on the peripheral normal skin, so that atrophy and achromasia or loss of pigmentation of true skin, or steroid acne, is disadvantageously caused to occur on such areas of the skin. Further, when the administration of the hormone is interrupted, in order to avoid adverse effects of the drugs, withdrawal dermatitis is often caused so that the lesion is likely to expand and deteriorate. Such withdrawal dermatitis is caused particularly when the administration of an internal preparation is discontinued. Accordingly, when the lesion occurs on a relatively large area of skin, the disease cannot be completely cured by this method alone and, therefore, this mode of therapy must be combined with other therapies.

As is evident from the preceding discussion, topical corticosteroids have setbacks, which may limit their use. In addition to the foregoing, one of the major problems with topical corticosteroid therapy is the ever-increasing cost of these medications. A 45 gram tube of any commercially available preparation may cost well over $100.00 and may be just enough to treat the extremities for a period of two weeks.

Common side effects from overuse or misuse of steroids include thinning of the skin, easy bruising and stretch marks. In addition, systemic absorption is always a risk as it can suppress the pituitary-adrenal axis and induce cushingoid features. All topical steroids have anti-inflammatory, anti-pruritic, and vasoconstrictive effects. Moreover, steroids are not recommended for use on the face. Therefore, a physician should monitor long term use of cortisone steroids.

As discussed above, it is difficult to treat psoriasis effectively by the use of adrenocortical hormone alone. With respect to the other therapeutic methods, such as the photochemotherapy and therapy using an epidermal cell growth inhibitor such as coal tar, anthralin, methotrexate, and retinoid, when these methods are used in combination with adrenocortical hormone, a therapeutic effect may be attained to some extent, but the psoriasis cannot be truly cured.

5.1.2 Coal Tar

Coal tar contains literally thousands of different substances and the exact mechanisms of action are unknown, though an antimitotic effect has been ascribed to these substances. Topical coal tar preparations have been used for centuries to treat the scaling, inflammation, and itching of psoriasis. Coal tar, depending on the strength, can be obtained as an OTC product, or by prescription. OTC products with tar concentrations of 0.5% to 5.0% are approved by the U.S. Food and Drug Administration (“USFDA”) for treating psoriasis. For example, Exorex™ is a new OTC preparation. The active ingredient in the Exorex Penetrating Emulsion is 1% coal tar. Tar shampoos are very often effective for scalp psoriasis.

Tar can be used by itself, and it is often combined with UV light B (UVB). Coal tar may make the skin more sensitive to UV and sunlight and cause photosensitivity, so extreme caution is advised when tar is combined with UV therapy or sunlight. This is important to remember for psoriasis patients with mild cases that are using coal tar products without the supervision of a doctor. A patient may be more susceptible to sunburn and a severe burn can occur. Thus, this should be considered when sun bathing, using a tanning booth, or receiving UV light therapy.

Common complaints about coal tar ointments are that they are messy, have an unpleasant medicinal odor, and can stain the skin and clothing. In addition, some people may find certain products irritating to the skin. Frequently tar applications are used in special protocols with light therapy units, which require a special clinical setup in which patients attend and spend most of the day for several weeks receiving treatment in order to clear the skin from the psoriatic plaques. This is a cumbersome ordeal to comply with, and not easily followed by most patients. In addition, it is extremely expensive with a two to three week treatment protocol running into the thousands of dollars. Finally, tars contain benzopyrene derivatives, which are known to be carcinogenic. For this reason, at least sixteen states, including California, have prohibited the use of tar shampoos without a prescription. Coal tar suppresses the growth of cells so that the lesion is diminished over a short period of time and a relatively long remission period may be achieved. However, occasionally, stimulant dermatitis and folliculitis (tar acne) may be caused.

5.1.3 Calcipotriene

Calcipotriene is a synthetic vitamin D₃ analog used for treating mild to moderate psoriasis. It constitutes one of the most recent formulations to be introduced for the treatment of localized psoriasis. Calcipotriene was first approved in the early 1990's in Europe as a 0.005% ointment preparation. In clinical trials, it is considered to have an effect comparable to class 3 corticosteroids. It is not as effective as the superpotent topical steroids, but it has been used in combination with these with an added effect. Sold as Dovonex in the U.S., this prescription medication is available in a cream, an ointment, and a scalp solution. It is not known for working quickly, thus there is a cost factor to this approach, since it may be several weeks before seeing improvement, but, generally, it is effective and safe for long-term control of psoriasis, with few known side effects. However, the lesions will reappear when use is discontinued. Moreover, the drug is not recommended for treating psoriasis on the face, can cause temporary skin irritation particularly over skin fold regions, and has been occasionally associated with hypercalcemia in some patients who apply large amounts of the medication. In addition, calcipotriene is significantly more expensive than topical corticosteroids.

To avoid the medication being absorbed internally, people are advised not to use more than 100 grams of Dovonex cream or ointment or 60 milliliters of scalp solution in a week. Dovonex is often prescribed in combination with other therapies, including topical steroids and UVB.

5.1.3 Vitamin A

Retinoid derivatives alter the delayed hypersensitivity response and increase the number of Langerhans cells in the psoriatic lesion. Tazarotene is a prescription topical retinoid (or vitamin A derivative) approved for treating mild to moderate plaque psoriasis. Tazarotene is particularly useful for psoriasis involving the scalp. For plaque psoriasis, retinoids can be used in combination with UV phototherapy to minimize the dose of each. Sold in the U.S. under the brandname Tazorac, this medication is available in a gel in two strengths: 0.1% and 0.05% and a cream formulation may soon be available. It only needs to be applied once per day, and Tazorac can be used to treat scalp psoriasis and nail psoriasis, as well.

Tazorac can be prescribed by itself, but more and more dermatologists are prescribing it in combination with a topical steroid. The results are better, and the side effects are reduced, particularly the moderate to severe skin irritation that Tazorac can frequently cause. The drug may also cause the psoriasis plaque to turn red before it clears, but this is a normal reaction and will go away. Contact to the eyes, eyelids, and mouth must be avoided. This medication should be avoided or discontinued immediately in pregnant women. Exposure to sunlight should be avoided and a sunscreen and protective clothing should be worn when outdoors. This product cannot be used with other photosensitizes. Tazarotene is extremely expensive. For this reason, and its tendency to irritate the skin, it has limited use in patients with psoriasis.

5.1.4 Anthralin

Anthralin is a synthetic derivative of a tree bark extract and an antiproliferative agent that may upset oxidative metabolic processes, decreasing the rate of epidermal cell proliferation. It is a prescription topical medication that has been used to treat psoriasis for more than 100 years. Anthralin is available in cream form and as a solution for scalp psoriasis, and physicians and pharmacists can compound stronger formulations. Anthralin can be very effective for mild to moderate psoriasis, with the important added benefit of having no long-term side effects. It is often used in combination with UVB treatments for more severe psoriasis. However, the medication does have two key drawbacks. It can be irritating to the skin surrounding psoriasis lesions, and it has the tendency to stain anything it comes into contact with, including skin, clothing, linens, and bathroom fixtures. Through special treatment regimens, the irritation and staining can be managed. Although anthralin is considered one of the most effective agents available, these problems have limited its use particularly in the U.S. Photosensitivity is also a concern if mixed with other psoriatic preparations. Anthralin should not be used by pregnant women. Anthralin can be combined with prescription steroids, coal tar, and moisturizers to create a treatment regime.

5.1.5 Salicylic Acid

In strengths of 1.8% to 3%, salicylic acid is approved by the USFDA as an OTC treatment for psoriasis. It is a keratolytic, which means it is used to soften and remove scales from psoriasis plaques. Removing scales is important, because it allows topical medications to reach and penetrate the skin. Salicylic acid is available in many forms, and it is often combined with other topical medications to enhance their effectiveness.

5.1.5 Sunlight

UV can clear or significantly improve psoriasis lesions for many people. The most effective wavelength, UVB, is found in natural sunlight. However, sunburn can cause psoriasis to get worse. In addition, sunbathing increases the risk of skin cancer.

5.1.6 Occlusion Therapy

Occlusion therapy involves covering the skin with an airtight, waterproof wrapping of a mild to moderate steroid or moisturizer. This enhances the penetration of the topical product and can help clear psoriasis, particularly stubborn localized plaques. Occlusion of steroids or other prescription medications should only be done under the supervision of a physician.

5.2 Phototherapy

Light treatments are one of the oldest therapies used for the management of psoriasis. The earliest form of this modality is the Goeckerman Regime, established in the 1930's, which combines the application of crude coal tar preparations followed by exposure to UVB lamps. This achieves a good and relatively prolonged remission. Over the years, new developments in UV lamps technology as well as improved clinical protocols have expanded its applications to other forms of UV light sources.

Phototherapy in general is used in treating patients with moderately severe and extensive psoriasis and patients showing resistance to topical treatments. It is expensive, time consuming, and may require the application of undesirable preparations such as tars and anthralin. Patients sometimes need to be hospitalized or frequently visit a phototherapy center to have their skin treated. There are two main forms of phototherapy; UVB and PUVA. When ultraviolet irradiation is carried out for a long period of time, not only is accelerated aging of the skin likely to occur, but also carcinogenesis may be induced.

5.2.1 Ultraviolet Light B (UVB)

UVB uses light in the 290-320 nm wavelengths and is usually combined with one or more topical treatments including coal tar followed by UVB (Goeckerman regimen), tar bath followed by UVB followed by anthralin (Ingram method), or UVB combined with topical corticosteroids, calcipotriene, tazarotene, or simply bland emollients. A major drawback is the time commitment required and accessibility to UVB equipment.

This type of treatment involves exposing the skin to a particular wavelength of ultraviolet light called UVB. It is a common, safe, and very effective treatment for moderate to severe psoriasis or localized areas of stubborn plaques. It may be used alone or in combination with topical or systemic treatments. Usually, three to five treatments per week for one to two months are required for most patients to reach clearing. A maintenance schedule of one or two treatments a week may help prolong remissions. Most people get UVB treatments at doctors' offices or clinics. However, under a doctor's supervision, a person can get a prescription to purchase a home UVB unit. It must be noted, though, that home phototherapy is a medical treatment that must be monitored by a physician.

There are two types of UVB treatment generally found in the United States: broadband and narrowband. The main difference is in the wavelength of light that is emitted by the equipment. Broadband UVB has been around for a long time and is more common in doctors' offices and in home phototherapy units. Narrowband UVB is newer, but it is becoming more widely available.

5.2.2 PUVA

PUVA involves the use of a prescription medication called psoralen and exposure to UV light A-hence the acronym PUVA. The difference between UVB and UVA (wavelengths in the 320-400 nm range) is the length of the light wave. It is also called “photochemotherapy.” The drug psoralen, which is taken by pill or applied topically to the skin, makes the skin more sensitive and responsive to this particular wavelength of UV light. The UV dosage is administered in the doctor's office and carefully monitored for the amount of exposure. There is a limit on the total exposure time that can be administered. Protective glasses must be worn to reduce the risk of cataracts. The psoriasis tends to return when discontinued. There is a significant cost per session and patients may need 3 to 5 sessions per week. PUVA clears or dramatically clears psoriasis for more than 75% of patients and can lead to extended remissions. However, due to the risks involved, PUVA is recommended only for moderate to severe psoriasis or disabling psoriasis when other treatments don't work. Long-term PUVA therapy can lead to premature aging of the skin, the forming of a peculiar lentigo or pigmented patch on the skin, and increase a person's risk of developing squamous cell skin cancer. The level of risk is related to several factors, including the patient's skin type, the number of treatments, and the cumulative “dose” of UVA administered to the skin. Other potential adverse effects include nausea, pruritus, burning, and photo damage to the skin. Recent articles have also revealed an increased risk for malignant melanoma, especially among patients who receive over 250 treatments after 15 years since initiation.

Psoralens, which are known to be mutagenic and carcinogenic, are components contained in many cosmetic preparations as aids to skin care. These photosensitizers may promote further skin reactions to UV radiation and result in permanent skin damage, cutaneous aging and malignancies even in patients with psoriasis.

5.3 Systemic Medications

Most of the oral prescription medications available to the psoriasis sufferer have serious side effects and are only used in severe, persistent cases that require rapid results. Generally, systemic treatment is initiated only after both topical and phototherapy have failed or for patients with very active psoriasis or PA. The main agents available are the immunomodulators Methotrexate and Cyclosporine and the oral retinoid Acitretin.

5.3.1 Methotrexate

Folic acid is active in inhibiting the growth of cells. Methotrexate, a drug originally to treat cancer, is a folic acid antagonist that inhibits DNA synthesis in tissues with high rates of turnover, such as psoriatic plaques, and is immunosuppressive to mononuclear cells in the skin, blood, and lymphatics. It is the most commonly prescribed systemic medication for severe and disabling psoriasis. It is taken orally or given by injection once per week, in a single or split dose. Methotrexate has a long history of proven effectiveness in clearing or greatly improving psoriasis, including erythrodermic and pustular forms of the disease. However, due to the possible risks associated with it, extremely careful monitoring is required, and should only be used under a physician's supervision. Short-term side effects include nausea, fatigue, loss of appetite, and mouth sores. Methotrexate has toxic effects on hematologic, renal, gastrointestinal, pulmonary, and neurologic systems. Thus, long-term side effects may include liver and lung damage, fetal death and birth defects, decreased numbers of blood cells, and intestinal ulcers. Prolonged usage may require annual liver biopsies to verify that the drug is not damaging the organ. In addition, methotrexate should not be used by pregnant women or men or women who are trying to conceive for several months prior to conception. It must be noted that many sufferers of psoriasis can fail to respond after prolonged use of methotrexate, and the disease may then rebound with an even greater severity. This treatment can be quite costly.

5.3.2 Oral Retinoids

The retinoid family of drugs is related to vitamin A. Acitretin (prescribed under the brandname Soriatane in the U.S.) is the only approved retinoid for treating severe cases of psoriasis. It is effective for pustular and erythrodermic types of psoriasis, but alone, it does not work as well on severe plaque psoriasis as some other drugs. Acitretin is also commonly prescribed in combination with other therapies, particularly UVB and PUVA. Isotretinoin (brandname Accutane) is an oral retinoid best known for treating severe cystic acne, although it is prescribed in some cases of severe psoriasis. However, this medication is not as effective as acitretin.

Tegison is an oral retinoid used for difficult cases. It can cause rashes, hair loss, and hepatitis. This drug is also known to cause birth defects and should not be used by women of childbearing age. It has been known to remain in the system for up to three years. In addition, Tegison is a very costly treatment.

The use of oral retinoid monotherapy has shown limited efficacy for chronic stable plaque psoriasis. Retinoids are considered to have an immunomodulation effect, that is, it may control the abnormal cornification of epidermal cells and the hyperfunction of leukocyte migration. People taking oral retinoids should not take vitamin supplements containing vitamin A, and those taking acitretin should avoid alcohol. The internal administration of retinoids, such as etretinate, is particularly effective for treating pustular psoriasis and psoriatic erythroderma. However, retinoids often exhibit an adverse effect wherein the thickness of skin and visible mucous membrane become small. Further, abnormal levels of serum lipoprotein are occasionally observed. Moreover, retinoids are teratogenic and likely to accumulate and remain inside the body for a long period of time and, therefore, the application of retinoids to a person capable of childbearing is to be avoided. For this reason, retinoids are usually applied only to patients who are beyond childbearing age or who are suffering from intractable psoriasis.

5.3.3 Cyclosporine

This drug, which suppresses the immune system, was originally developed to prevent the rejection of transplanted organs. Cyclosporine is approved for treating severe psoriasis in those people who cannot take or have not responded to other systemic therapies. It is available in either capsule or liquid form, which must be diluted for use. Cyclosporine generally works very well at improving or clearing psoriasis, and it is considered quick acting. Results may be seen in as little as two weeks. However, cyclosporine has the risk of causing high blood pressure and damaging kidney function, and while these side effects are usually reversible if treated promptly, they are real and potentially serious. Another adverse effect is the potential increased risk of cancer. This medication is not approved for continuous treatment of more than one year. Cyclosporine inhibits production of interleukin-2, the cytokine responsible for inducing T-Cell proliferation. Skin lesions recur within days to weeks after treatment is stopped.

5.3.4 Other Medications

Several other systemic medications have been used over the years for treating severe psoriasis, but they are prescribed less often than the drugs mentioned above. Hydroxyurea, sulfasalazine, 6-thioguanine, and mycophenolate mofetil have all shown some effect in improving psoriasis. Each has its own risks and side effects, and none has been approved for treating this disease.

5.4 Other Approaches

5.4.1 Combination Therapy

When psoriasis is resistant to one therapy, a combination of treatments may be the answer. Such a combination can result in quicker response and may reduce the side effects. Examples include prescribing a low dose of acitretin in combination with UVB or PUVA, or using tazarotene in combination with a topical steroid. However, possible combinations of treatments that could work vary for each individual case of psoriasis.

5.4.2 Rotational Therapy

All of the therapies used for treating moderate to severe psoriasis have risks and side effects. Rotating these treatments can minimize a patient's exposure to the toxic properties of these therapies, and people may avoid becoming resistant to certain treatments. Five therapies used in rotation for extensive psoriasis are tar plus UVB, PUVA, methotrexate, acitretin and cyclosporine. One treatment is used for 12 to 36 months, and then another is used, and so forth.

6. PA Therapies

Current therapies for PA can relieve pain, control skin lesions and joint inflammation, and possibly prevent further tissue damage. Physicians will choose treatments based on the type of PA, its severity and an individual's reaction to treatment. It is important for people who seem to be developing severe PA to begin appropriate treatment before irreversible changes occur. Early treatment can help slow the disease and preserve function and range of motion. Some early indicators of severe disease include onset at a young age, spinal involvement, and the results of certain blood studies.

Pharmacological therapy is similar to that used for rheumatoid arthritis, and it is mainly based on the use of nonsteroidal anti-inflammatory drugs (NSAIDs) or steroids. Such drugs exert an effective action on flogosis and pain, but they should be used with extreme caution since, in addition to their well-known side effects, they may cause skin lesions to exacerbate and become pustular.

Other drugs used in the treatment of PA induce adverse side effects as well. For instance, Penicillamine exerts beneficial effects similar to those of gold compounds, but it may induce side effects requiring discontinuation, such as marrow suppression, nephrosis, and proteinuria. Cytotoxic or immunosuppressive drugs, such as Azathioprine and Methotrexate, may only be used in severe cases of the diseases, since they induce major side effects, such as bone marrow suppression, liver disease, pneumonitis. Etretinate may be effective in severe psoriasis, but it can induce hypervitaminosis A, teratogenicity, and hepatic toxicity.

Drugs for the treatment of PA can be divided into two categories:

i) NSAIDs, including OTC medications such as aspirin and ibuprofen as well as prescription products; the main purpose of these medications is to decrease the symptoms of PA, including inflammation, joint pain, and stiffness.

ii) Disease-modifying antirheumatic drugs (DMARDs), whose purpose is to relieve more severe symptoms and attempt to slow or stop joint and tissue damage and progression of PA.

Corticosteroids play a helpful role in some cases. Heat, warm water soaks, exercise programs, and physical therapy also are used in the treatment of PA.

6.1 Aspirin & NSAIDs

Aspirin is often less effective for PA than for rheumatoid arthritis, even though it can help reduce pain, swelling and stiffness. Aspirin can cause bleeding in the stomach, ulcers and easy bruising. Prescription and nonprescription NSAIDs are effective for many people with PA in controlling swelling, pain and morning stiffness, and in improving range of motion to joints. They can help reduce the limitations to daily activities often caused by arthritis. Many different brands of NSAIDs are available. The specific drug to be used is determined between the individual person with PA and his or her physician. It may take some experimentation before the NSAID with the right combination of good results and low side effects is found. NSAIDs and aspirin generally do not significantly alter psoriasis skin lesions, although certain NSAIDs have been reported to trigger flares of psoriasis. NSAIDs and/or aspirin are sufficient treatment for many people over time. Acetaminophen (Tylenol) may be added for pain relief. A physician considers stronger medications when NSAIDs and aspirin fail to work and progression of the disease is evident. Some NSAIDs, when taken in high doses or over long periods of time, carry a risk of causing stomach problems, including ulcers and gastrointestinal bleeding. The risk depends on the strength of the NSAID and how long it is taken, and many people with PA do not have problems with NSAID side effects. For those who have had stomach problems or are at a higher risk for them, a new type of NSAID called a COX-2 inhibitor is now on the market. It has been developed to be safer for the stomach than other NSAIDs. The COX-2 inhibitor celecoxib (brandname Celebrex) has been approved for treating the symptoms of rheumatoid arthritis and osteoarthritis, while rofecoxib (brandname Vioxx) is indicated for symptoms of osteoarthritis and for acute pain. Celecoxib and rofecoxib are not necessarily more effective at relieving pain and inflammation than regular NSAIDs. They are more expensive and have their own risks.

6.2 Methotrexate

Methotrexate is used widely and successfully for treating PA and rheumatoid arthritis. It is effective at relieving the symptoms associated with PA, and it may retard the destruction caused by certain forms of PA. Methotrexate usually is well tolerated in low doses. It does, however, have a number of side effects, as discussed above, and the long-term potential of damaging the liver. With careful management and dosage, the drug can be used safely by selected individuals for years.

6.3 Cyclosporine

As previously discussed, cyclosporine is approved for treating psoriasis, and it has produced improvement in the skin and in joint disease. Use of cyclosporine has increased recently, and it may be combined with methotrexate in certain individuals. Frequent blood tests are required due to the possibility of kidney damage. In addition, the drug can cause high blood pressure, and increases the risk of developing lymphomas and other types of cancer. It is a costly treatment.

6.4 Sulfasalazine

Sulfasalazine, a sulfa drug developed to treat inflammatory bowel diseases, has been increasingly used for PA as well as rheumatoid arthritis. Approximately one-third of PA patients respond rapidly to this therapy (usually within four to eight weeks), which may also induce more sustained remissions of the disease. This drug has less dangerous side effects than some other systemic psoriasis and PA treatments, including methotrexate, so a trial of sulfasalazine may be worthwhile for some. However, many people cannot tolerate sulfasalazine because of side effects, including nausea, vomiting and loss of appetite. Sulfasalazine is quite effective, but it may cause neutropenia, hemolysis and hepatitis.

6.5 Antimalarials

Antimalarial therapy, commonly used with success in rheumatoid arthritis, has sometimes been used to treat PA. However, certain antimalarials can cause skin psoriasis to get much worse in some people. Some experts think antimalarials should not be used at all for PA.

6.6 Retinoids

Acitretin may be effective for some PA patients. Oral retinoids carry with them the risk of birth defects and the possibility of producing skeletal side effects with long-term use.

6.7 PUVA

PUVA therapy may sometimes improve PA affecting the limbs. Generally it is used in combination with other medications. It is not helpful in treating PA of the spine. It has few short-term side effects, but it has the long-term potential to increase the risk of certain skin cancers. The amount of risk is based on several factors, including the individual's skin type, the number of treatments, and the total “dose” administered.

6.8 Gold (Chrysotherapy)

Injection of gold salts and administration of gold capsules by mouth have both been reported to be effective in treating arthritis affecting the limbs, but not for treating arthritis of the spine. Though some people report that it causes a rash, which makes psoriasis worse, others say it seems to improve their psoriasis lesions. Blood and urine samples are required to prevent kidney damage. Use of gold has declined somewhat in recent years as new therapies have been developed. Gold compounds are somewhat beneficial, but they may cause toxic effects and are contraindicated in patients with hepatic or renal disease.

6.9 Azathioprine

Azathioprine (brandname Imuran) is a drug that suppresses the immune system, and it is approved for use in certain types of arthritis. It has potent anti-inflammatory effects. Blood tests must be performed frequently because the drug can cause life-threatening effects on the bone marrow. Azathioprine increases the risk of malignancies in later years.

6.10 Corticosteroids

Steroid medications taken orally are not generally recommended for long-term treatment of PA, although in some circumstances they may be needed for relief of acute, severe joint inflammation and swelling. For the most part, large doses of steroids injected into muscles should be avoided-psoriasis of the skin may become much worse when treatment with systemic steroids is stopped. Occasionally, severe forms of psoriasis, such as pustular psoriasis, may be provoked by the use of systemic steroids. However, selective low-dose steroid injections to inflamed joints, tendons and the area around joints can improve range of motion and limit contraction.

6.11 New Therapies Being Developed

Several new therapies approved by the USFDA for treating rheumatoid arthritis are proving beneficial for PA as well. These therapies, called “biologic response modifiers,” target the immune system response that leads to inflammation. They represent an exciting and promising area of research for the treatment of inflammatory forms of arthritis, including PA. In small studies and in anecdotal reports, etanercept (brandname Enbrel) has shown very good results in improving the symptoms of PA. There are very few side effects, although there can be an increased risk of infections. Large-scale testing of this drug in PA patients was underway in 2000, in preparation for application to the USFDA for approval. In the meantime, many physicians are already prescribing etanercept “off label” for their PA patients. Etanercept is given through a self-administered injection. Infliximab (brandname Remicade) is another biologic response modifier that is being used “off label” for PA in limited cases. This drug is approved for rheumatoid arthritis and Crohn's disease, an inflammatory bowel condition. Several small studies of the effectiveness of infliximab for treating PA and psoriasis are ongoing. Infliximab is administered by injection in a doctor's office. A third new rheumatoid arthritis drug is leflunomide (brandname Arava). It is similar to methotrexate. Leflunomide, which comes in a pill, is proving beneficial to some people with PA, according to anecdotal reports. These drugs may be very expensive (up to $1,000 per month), and insurance companies may not cover them because they are not USFDA approved specifically for PA.

6.12 Other Approaches

6.12.1 Surgery

Surgery can help people whose joint destruction limits motion and function despite medical therapy. (Skin affected by psoriasis does not appear to cause any special problems with infection during surgery).

6.12.2 Rehabilitation

Physical therapy and rehabilitation are used to maximize the function of an arthritic joint.

6.12.3 Exercise

Exercise is essential to preserve strength and maintain range of motion. Isometric exercise is often prescribed because it appears to be less damaging to inflamed joints. A range of motion program should be coupled with a stretching program. Stretching exercises are part of the treatment and are especially useful for spinal arthritis.

6.12.4 Splints

In addition to exercise and local pain therapy, a splint may be used to support a joint in a position to improve function and relieve pain and swelling.

6.12.5 Other

Heat, cold, and rest are used to relieve pain. Immobilizing an inflamed swollen area while using cold packs can reduce the swelling and improve range of motion. Osteoporosis may occur with arthritis, especially with psoriatic spondylitis, and fractures from minor trauma may occur. Calcium supplements, along with vitamin D, help prevent it in affected individuals. In addition, calcitonin and biphosphonates (prescription drugs that affect calcium metabolism and bone formation) may be used.

7. Alternative Treatments for Psoriasis and PA

Self-treatment and the use of alternative medicine are very common among people with psoriasis and PA. In some cases, they may be tired of the challenge of finding a traditional therapy that works, or they may be concerned about the possible side effects associated with many of those therapies. No matter the reason, the popularity of nontraditional treatment approaches is growing. In many areas of alternative medicine, however, there is little or no scientific evidence that these practices will improve symptoms of psoriasis and PA. There are stories involving just about everything one could imagine being used as a treatment. However, it must be noted that herbal, natural, alternative, and homeopathic approaches are not intended to replace common sense or a doctor's treatment plan.

7.1 Acupuncture

Acupuncture is a technique involving the manipulation of needles placed in the body. Needles are placed at specific “meridians” or acupressure points to relieve pain and treat disease. Several uncontrolled studies have shown that acupuncture is an effective treatment for psoriasis. Reports are anecdotal and mixed at best with some people saying they have been helped by it and others not. Nevertheless, people who have tried it and have had improvement say it takes many treatments.

7.2 Ayurveda

This ancient healing method originated in India more than 5,000 years ago. Prevention is the key to Ayurvedic medicine. It encourages attention to balance in one's life through diet, herbs, lifestyle, and right thinking. In Ayurvedic medicine, managing disease and restoring health involves assessing the whole person to understand the nature of the imbalance. For psoriasis, a specific treatment regimen might involve topical application of certain oils, such as sesame or mustard; fasting and other dietary guidelines; the elimination of stress; and regular physical exercise. However, no known studies specifically look at Ayurvedic medicine as a therapy for psoriasis.

7.3 Chiropractic

Chiropractic is the practice of manipulating the spine for therapeutic benefit. The practice began in the U.S. in the late 1800s and is now a widely recognized alternative therapy. Although it has been reported to work in conjunction with a strict diet and internal cleansing (enemas), the overall importance of chiropractic manipulations in the treatment of psoriasis has not been proven.

7.4 Climatotherapy

Sunlight can be a valuable treatment option for people with psoriasis. Eighty percent of the people who get regular daily doses of sunlight enjoy improvement or clearing of their psoriasis. Climatotherapy is a term sometimes used to discuss the use of sunlight and water, such as the ocean or other bodies of water, to treat psoriasis. For climatotherapy, the best-known location is the Dead Sea in Israel, where each year thousands of people with psoriasis and other skin diseases go for extensive sun exposure and baths in the unique water. This treatment is very effective and safe in most cases, although it is an expensive option for people from the U.S. Other climatotherapy sites do exist around the world, but their facilities and reputation do not match that of the Dead Sea.

The word “salt” does not refer only to sodium chloride, or table salt. Salts are a form taken by many minerals. The Dead Sea has the largest concentration of mineral salts in the world. Since ancient times, soaking in the intense mineral baths or “salt” baths has been recognized for its rich therapeutic qualities. Israeli dermatologist Dr. Zvi Even Paz studied the effect Dead Sea bath salts had on fifty psoriasis patients in 1989. Although it was long known that bathing in the Dead Sea would bring about temporary relief from this incurable condition, it was not known whether bathing in the Dead Sea salts anywhere would produce the same results. For forty-seven of the fifty patients tested, significant relief was achieved.

7.5 Diet

There is no specific diet that people with psoriasis should follow. Manipulating the diet has not been found to be useful for PA or psoriasis patients. The best diet is the one that makes the individual feel the best, because people with psoriasis benefit from a healthy lifestyle and eating habits, just like everybody else. Still, there is much anecdotal evidence that certain foods act as triggers to psoriasis. In turn, several dietary regimens have been put forward with claims that they will improve psoriasis. However, convincing scientific studies have not been conducted to verify these claims. Many people are controlling their psoriasis using the cleansing diet approach. However, diet and the impact of certain foods on psoriasis is understudied. Combining therapies, (example, diet with homeopathic minerals, diet and herbs, omega oils with diet or herbs) is showing positive results in greater numbers of psoriasis patients. For example, homeopathic minerals designed specifically for psoriasis have been shown to benefit mild to moderate cases of psoriasis. When combined with a cleansing diet, the benefits are increased.

7.6 Herbs and Herbal Treatments

Most drugs originate from plants or products in nature. Quinine, used for the treatment of malaria, was discovered by watching the natives eat the bark of the cinchona tree. Herbal remedies and ancient techniques are now surfacing and becoming popular. However, not everything that is natural is safe or good. An herb can have certain remedial benefits. These are well known and published. To keep herbs available for sale in the United States, they must be called “food supplements” and no claims can be made about their medicinal qualities.

7.7. Dietary Supplements

The category of “dietary supplements” covers a lot of ground—from vitamins to minerals to herbal products to a wide range of miscellaneous substances. In most cases, claims that a particular dietary supplement can improve or cure psoriasis have not been proven scientifically. However, clinical studies do exist on fish oil supplements and evening primrose oil, both of which produced some good results in psoriasis. Other supplements mentioned in various reference books include burdock, milk thistle, yellow dock, red clover, mountain grape, and sarsparilla. Taken in proper amounts, many supplements are probably harmless, but consumers are largely on their own in safeguarding their health. In addition, dietary supplements can interact negatively with prescription medications.

7.8 Homeopathy

Homeopathy is a 200-year-old healing art founded in Europe. It is a school of medicine based on the premise that “like cures like.” Practitioners believe that substances that produce certain symptoms in a healthy person can cure diseases that have similar symptoms. As an example, homeopathic physicians might use a natural product known to cause stomach irritation in a healthy person and administer it in a greatly diluted form (from three to 100,000 times) to treat a stomach ulcer. Homeopathy is gaining renewed popularity and recognition in the United States. Complementing rather than opposing traditional medicine, homeopathy offers a therapeutic alternative that is natural, safe, and effective. Low dose homeopathic ingredients help natural bodily mechanisms correct underlying disfunctions and relieve disease symptoms. Utilizing approved ingredients from the Homeopathic Pharmacopoeia of the United States, low dose minerals have shown promise in relieving mild to moderate cases of psoriasis and other diseases. However, evidence is mostly anecdotal and not scientific. Homeopathic remedies are so diluted that generally they will not cause any side effects.

7.9 Magnets

Promotion and use of therapeutic magnets for relieving pain has become more and more popular in recent years. While there are several theories as to why they might be effective, no one is absolutely sure how magnets work. It is easy to find positive testimonials about magnet therapy, but rigorous scientific studies are less common. Anecdotal reports of unpleasant side effects do exist, and magnets are not guaranteed to be safe.

7.10 Meditation/Relaxation

There is clear anecdotal and scientific evidence that stress can trigger or aggravate psoriasis in some people. Therefore, practices that promote relaxation and stress reduction are often recommended for people with psoriasis. Hypnosis, both on its own and as an addition to other psoriasis treatment, was cited as being helpful in clearing psoriasis in several published case reports. One study examined the use of meditation-based relaxation tapes in patients undergoing UV treatments. Patients who listened to the tapes during the treatments cleared faster—in some cases, twice as fast—than patients who had the light therapy only. Along those same lines, an unpublished study found that PUVA patients who had some type of psychological intervention or relaxation training improved much faster than patients who had no intervention. The mind can be a powerful tool for healing. Any technique that can help people with psoriasis learn to manage stress and give them a better sense of control over the mental aspects of the disease is potentially worthwhile. These techniques, however, seem to work best when used to supplement—not replace—traditional medical treatments.

7.11 Essential Fatty Acids

Within the psoriasis community, awareness about the benefits of using supplements of essential fatty acids, continues to emerge. Increasing nutrition and metabolism through increasing the body's ability to absorb vitamins, minerals, and amino acids all seem to play an important key role in decreasing the psoriasis condition.

7.12 Omega 3 and 6 Oils

The lack of the “good” oils in the modern diet is a major concern in alternative medicine circles. The lipids and amino acids necessary for healthy cells are not available, even in the best of diets, so diet supplements may help the body restore itself to a normal condition (normal being disease free). In some instances, just adding these essential oils has shown benefits. Oils (fats) are not all “fatty” and fat free diets can deplete the essential fatty acids necessary for proper utilization of vitamins and minerals. Flax is a plant that is gaining popularity as a rich source of omega 3 oil. It is relatively inexpensive, and most naturopaths and alternative approaches encourage some form of flax taken daily to maintain and promote health. Flax seed contains all 8 amino acids.

It is a well-known fact that fish liver oils are high in the omega 3 and 6 oils. Vitamins A and D, (both important to the skin and found in abundance in fish and fish liver oils) and Vitamins E and K are all fat-soluble, meaning they are not dissolved in water but in oils. A tablespoon of cod liver oil was a daily part of raising a healthy child long before children's vitamins became popular. But the omega polyunsaturated oils are only part of the nutrients available in fish.

7.13 Soy

Lecithin, long used by psoriasis sufferers, is a member of the family of fatty substances known chemically as phosphatidylcholine. It is available in capsules, or as a granular food supplement. Most of the lecithin available today comes from the soy plant. The role is phosphatidylcholine and choline in cell functioning is well documented scientifically. The role of lecithin in restoring normal skin or decreasing psoriatic lesions has not been determined scientifically (no studies have proven conclusively that this will or will not benefit the psoriasis sufferer).

7.14 Alkylglycerols (“AKGs”)

AKGs are a family of compounds that are integral in stimulating the body's immune system. They are found in significant amounts in human breast milk, the liver, spleen, and bone-marrow, all of which are agents of the immune system. As a natural supplement AKGs can also be found in shark liver oil. AKGs are active substances that give breast-fed babies crucial protection against infection until their own immune systems are fully developed.

7.15 100% Emu Oil

Emu's have been around for 80,000,000 years. The Australian Aborigines have been using emu oil for over a thousand years to help promote healing of almost every imaginable injury and ailment.

7.16 Miscellaneous Topical Products

Moisturizing the skin is an important part of treating psoriasis, and the market is full of moisturizing products that could fall into the “alternative” or “natural” category. Aloe vera was shown in one study to be effective in treating psoriasis. Neem oil, emu oil, jojoba, and other substances have no evidence that they help psoriasis, but they may because of their moisturizing properties. Bath and moisturizing products containing oat derivatives are known to be soothing to the skin. Other topical products may have more medicinal effects, such as witch hazel, tea tree oil, mahonia aquifolium, capsaicin, and evening primrose oil. Capsaicin (a natural ingredient in hot peppers) helped scaling, redness and itching in two studies on psoriasis, and it is also well known as a topical pain reliever. Several OTC capsaicin products are available.

7.17 Traditional Chinese Medicine (“TCM”)

TCM is an ancient health care system that involves many different practices. The administration of many herbs, combined in unique formulations for each individual patient, is one of the primary treatments. TCM can come in oral, topical, and injectable forms. In China, many herbal preparations, both oral and topical, are available for treating psoriasis. Some of these make the skin more sensitive to UV and are combined with traditional phototherapy. There are anecdotal reports of people with psoriasis improving on TCM, but studies are difficult to conduct because the herbal mixtures are often individualized.

8. Summary

Psoriasis is a life-long disease, which requires the use of multiple modalities of treatments to achieve a significant relief. Most current treatments for psoriasis act by regulating the immune system or otherwise attenuating the inflammatory response. It must be noted, however, that internal medications such as cyclosporine, methotrexate, and retinoids all have potentially serious side effects such as liver and kidney damage, nausea, birth defects and increased cancer risk. Other common psoriasis treatments are also undesirable for long-term management of the disease. For instance, extended use of topical corticosteroid creams may cause thinning of the skin, stretch marks, and suppression of the patient's own cortisol production. Moreover, psoriatic symptoms tend to recur rapidly after cessation of corticosteroid use. In turn, phototherapy can result in skin aging and increased risk of skin cancer.

Thus, the need for an effective treatment, which does not induce severe side effects, as those mentioned above, is quite evident. The present invention has the benefit of combining well-known, safe, and effective active ingredients, which have been formulated in an easy to apply lotion that clears the disease in a short period of time without any known side effects and is significantly less expensive than the commercially available alternatives.

As discussed above, several topical treatments are available. However, no single ideal topical agent exists for plaque psoriasis. The composition and method of the present invention will change that. The present invention provides a topical composition that has the ability to internalize rapidly into psoriatic tissue and inhibit virtually every aspect of psoriasis.

Therefore, it can be appreciated that there exists a continuing need for a new and improved topical treatment for psoriasis. In this regard, the present invention substantially fulfills this need. The present invention overcomes the inability of prior art to provide a single topical agent for treatment of the different forms of psoriasis.

Various novel therapies for psoriasis have recently been disclosed, yet none provide a faster, cost efficient, healing with no side effects as the composition of the present. For instance, U.S. Pat. No. 5,776,920 to Quarles, teaches a preparation of salicylic acid, lactic acid, and urea in a moisturizing medium useful as topicals for psoriatic lesions applied once daily for four days. U.S. Pat. No. 5,719,195 to Braiman teaches the use of isomers of retinoic acid, namely II-cis-retinoic acid, as a therapeutic agent. U.S. Pat. No. 5,648,373 to Winkler et al teaches the use of enzyme inhibitors to block the production of inflammatory mediators (the arachidonic acid cascade). Furthermore, U.S. Pat. No. 5,714,505 to Hasselkus discloses the use of omeprazole in the therapy of these skin maladies. U.S. Pat. No. 5,565,462 to Eitan et al teaches the use of xanthine derivatives, namely, pentoxifylline, propentofylline and torbarylline, as topicals for psoriasis and atopic dermatitis. U.S. Pat. No. 5,783,596 to Medford teaches the use of dithiolarxy-lates, especially dithiocarbamates as therapies for inflammatory diseases by blocking the induced expression of the endothelial cell surface adhesion molecule VCAM-1.

U.S. Pat. No. 6,346,251 to Zamoyski discloses compositions of sesquiterpene epoxide compound (trichothecenes) and methods for treating psoriasis. U.S. Pat. No. 6,333,027 to Hopkins, et al. Discloses a composition for treating psoriasis and other skin disorders containing several surfactants and an active ingredient. U.S. Pat. No. 6,113,893 to Burnett, et al. discloses a topical composition for the treatment of psoriasis. U.S. Pat. No. 6,107,349 to Mantynen discloses a method and composition for treating psoriasis, which combines Vitamin E, evening primrose oil, and B-complex vitamins. U.S. Pat. No. 6,011,067 to Hersh discloses an antioxidant composition for the treatment of psoriasis and related diseases. Hersh teaches that the synergistic antioxidants of his invention may be combined with anti-inflammatories, including corticosteroids, anti-microbials, including zinc pyrithione, and other preparations useful in the therapy of desquamating disorders as psoriasis, seborrhoeic dermatitis and related skin and scalp conditions.

However, none of the art considered above, taken either simply or in combination teach the use of triamcinolone acetonide, polysorbate 80, and zinc pyrithione for treating psoriasis and related conditions.

### SUMMARY OF THE INVENTION

The object of the present invention is to provide a new pharmaceutical composition and a method for its use in the topical treatment of psoriasis and related skin disorders that clears the eruptions of psoriasis in the skin.

Another object of the present invention is to provide a new pharmaceutical composition and a method for its use in the topical treatment of psoriasis and related skin disorders that eliminates or reduces the scaling and discomfort due to the disease.

It is a further object of the present invention to provide a composition for the topical treatment of psoriasis and a method for its use that restores normal skin growth for extended periods without recurring symptoms.

It is also an object of this invention that the treatment avoids the drawbacks of known treatments for psoriasis. In particular, it is an objective of the invention to eliminate undesirable systemic effects, tachyphylaxis or tolerance buildup and side effects, which can result from known treatments.

It is a further object of the present invention to provide a treatment for fungal and bacterial growth in different parts of the body prone to such growth including the feet and toes.

Another object of the present invention is to provide a topical lotion that is easy to use and apply with minimal training.

Another object of the present invention is to provide a topical solution that can be used to treat all parts of the body.

A further object of the present invention is to provide a compounded medication formula for treating skin disorders to essentially clear the scales, plaques, redness, inflammation, and leave the skin with healthy smooth appearance.

It is still a further object of the present invention to provide a compounded medication formula which obtains the desired results even after washing, or sweating in the treated area.

Another object of the present invention is to provide a topical solution that is of low cost.

It is a further object of the present invention to provide an efficient treatment with low wasted time.

It is an object of the present invention to provide a method for treatment of psoriasis or other undesirable skin disorders which is safe.

Still another object of the present invention is to provide a method for treatment of psoriasis or other undesirable skin disorders which reduces and/or eliminates “itchiness” of skin which is a common symptom/condition of psoriasis or skin disorders.

Still another object of the present invention is to provide a method for treatment of psoriasis or other skin disorders which reduces and/or eliminates the undesirable “redness” appearance of skin which is a common symptom/condition of psoriasis or of skin disorders.

Still another object of the present invention is to provide a method for treatment of psoriasis or other skin disorders which reduces and/or eliminates undesirable skin scales which is a common symptom/condition of psoriasis or skin disorders.

Still another object of the present invention is to provide a method for treatment of psoriasis or other skin disorders which slows moisture loss of affected skin and tissue which is a common symptom/condition of psoriasis or skin disorders.

Still another object of the present invention is to provide a method for treatment of psoriasis or other skin disorders which facilitates the individual layers of skin to return to a normal rate of growth and elevation for the treatment of the skin disorder.

Still another object of the present invention is to provide a method for treatment of psoriasis or other skin disorders which minimizes relapse or recurrence of the skin disease following completion of the treatment regime.

Still another object of the present invention is to provide a method for treatment of psoriasis or other skin disorders which returns moisture to areas of skin affected with the undesirable skin disease.

The composition and method themselves, both as to their construction and mode of operation, will be best understood, and additional objects and advantages thereof will become apparent from the following description of the preferred embodiments.

When the word “invention” is used in this specification, the word “invention” includes “inventions”, that is, the plural of “invention”. By stating “invention”, the Applicants do not in any way admit that the present application does not include more than one patentably and non-obviously distinct invention, and Applicants maintain that the present application may include more than one patentably and non-obviously distinct invention. The Applicants hereby assert, that the disclosure of the present application may include more than one invention, and, in the event that there is more than one invention, that these inventions may be patentable and non-obvious one with respect to the other.

Further, the purpose of the accompanying abstract is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

### DESCRIPTION OF THE PREFERRED EMBODIMENT

The present invention encompasses a new composition and a method for its use in the topical treatment of psoriasis and related skin disorders. The composition of this invention achieves the synergetic regeneration for skin disorders with the active ingredients of triamcinolone acetonide, Zync Pyrithione, and Polysorbate 80 with no side effects.

In the preferred embodiment, the composition according to the present invention comprises from about 0.01 to about 5.0 weight percent zinc pyrithione, from about 0.01 to about 5.0 weight percent triamcinolone acetonide, from about 0.01 to about 10 weight percent polysorbate 80, from about 5 to about 45 weight percent isopropyl myristate, from about 5 to about 45 weight percent ethyl alcohol, from about 2 to about 30 weight percent purified water, from about 0.01 to about 10 weight percent sodium lauryl sulfate, from about 0.01 to about 5 weight percent vitamin E, from about 0.01 to about 5.0 weight percent of aloe vera, from about 0.01 to about 5 weight percent vitamin D3, and from about 0.01 to about 3 weight percent undercylenic acid.

Zinc pyrithione has been used in a number of dermatologic preparations, including shampoos, sprays, creams and lotions. It is available in commercial preparations for management of dandruff, seborrhoeic dermatitis, flakes (as in psoriasis) and other skin maladies. Purportedly, this zinc salt possesses antibacterial and antifungal properties, particularly against pityrospora species present in seborrheic dermatitis scalps.

The medical literature has several reports of the beneficial use of zinc pyrithione alone in psoriatic patients. For example, U.S. Pat. No. 4,323,558 to Nelson teaches pharmaceutical compositions containing triethylenetetramine (Trien) for use in various inflammatory skin disorders. The inventor adds that Trien may be used in gels, ointments, and lotions even with inclusion of zinc pyrithione with which Trien forms a clear solution of gel.

Isopropyl Myristate is one of the best vehicles for penetration of the skin and mixes well with the anti-inflammatory triamcinolone acetonide. In turn, Polysorbate 80 also mixes well with triamcinolone acetonide, has humectant's properties, and mixes with water easily keeping the skin moist and enhancing the skin appearance emidially. Polysorbate 80 is a form of Sorbitol and when and when combined with Isopropyl Myristate, Undercylenic acid, and Zinc Pyrithione loosens the dry silvery skin plaques which appear in the skin surface of the extremities, scalp, trunk, and back, and emulsifies the waxy build scales on the scalp. In alternative embodiments of the invention, other vehicles of the active ingredients may be soaps, shampoos, creams, sprays, unguents, balms, ointments, and sticks.

An alternative embodiment of the invention is comprised of from about 0.01 to about 5.0 weight percent Zinc Pyrithione, from about 0.01 to about 5.0 weight percent of a corticosteroid, from about 0.01 to about 10 weight percent Polysorbate 80, from about 0.01 to about 5.0 weight percent of an anti-fungal or anti-bacterial agent, and from about 0.01 to about 45% weight percent of at least one medication carrier. Another alternative embodiment is possible by adding at least one anti-oxidant to the aforementioned group of components.

Topical corticosteroids are synthetic derivatives of cortisone that when applied to skin affected by psoriasis will effectively control inflammatory, allergic, and pruritic dermatoses. The fluorine atoms introduction into the molecule enhances the anti-inflammatory effectiveness. Triamcinolone acetonide is the corticosteroid used in the preferred embodiment of this invention. It is classified as either a class 3 or 4 steroid depending on its vehicle formulation. Triamcinolone acetonide is a safe medication used to treat asthma, and as anti-inflammatory in skin creams, and balms sold under brand names such as Kenelog, Asmacor, Aristocor A, Tri-Nasar, and Nasacort AQ. The application of triamcinolone acetonide acts by controlling the rate of synthesis of the proteins. The pharmaecokinetics of a topical application to the normal skin of triamcinolone acetonide is minimally absorbed, and low concentration of the medication penetrates to the dermis.

Examples of suitable medication carriers for alternative embodiments include isopropyl myristate, ethyl alcohol, purified water, and sodium lauryl sulfate. Nevertheless, in other embodiments of this invention the active ingredients described above can be incorporated in any suitable pharmacologically acceptable carrier, which is suitable for topical administration to the human skin. As such, the pharmacologically acceptable carrier must be of sufficient purity and have sufficiently low toxicity to render it suitable for administration to a human noting that the carrier can represent at least approximately 80% of the total composition. Typical compositions for use herein include a wide variety of physical forms. These include, but are not limited to, solutions, lotions, creams, oils, gels, sticks, sprays, ointments, balms, shampoo and pastes. Generally, such carrier systems can be described as being solutions, emulsions, gels, solids and aerosols.

Examples of suitable antioxidants for alternative embodiments include vitamin E, aloe vera, and vitamin D3. The anti-oxidants Vitamin E, and Aloe Vera are used for skin nourishment. Vitamin E, particularly in its alpha-tocopherol moiety, has been employed to inhibit oxidation of oils and fats in foods, cosmetic preparations and drugs. Vitamin E is not only an antioxidant but also has anti-inflammatory properties. In skin, vitamin E levels are present in dermis and epidermis, but are depleted by malnutrition and by UV light, thus their importance too in providing these to act in vivo as antioxidants, elevating the UV exposed tissue levels and thereby protecting affected skin cells. Vitamin E moisturizes and enhances skin smoothness. It is soothing and also participates in skin repair and wound healing, such as occurs in photoaging and sunburn and required in psoriasis treatments.

An example of a suitable anti-fungal or antibacterial agent for alternative embodiments is undercylenic acid.

The absorption of medication is lower in patients with psoriasis. However, the medication carriers of the preferred embodiment of the invention enable all the medication to be carried deep into the skin to the dermis, solvents evaporating lining Polysorbate 80 as a film in the skin Psoriasis silvery plaques. Greater absorption of the medication is achieved in body areas like the face, eyelids, and genitals. Absorption is decreased by other carrier vehicles, plaques, and scales in the disease epidermis. The topical compounded medication therapy of this invention produces fast responsive enhancement when required by skin chronic eruptions, where anti-inflammatory, anti-allergenic, anti-bacterial, anti-fungal, and anti-pruritic activities are noticed.

The invention uses triamcinolone acetonide because of its long history of no complications when used in prolongued therapy in asthmas sprays used twice a day. Fewer side effects and low toxicity will be noticed with this invention because of the use of triamcinolone acetonide and other active ingredients in low concentrations.

The Undercylenic acid provides anti-fungal protection of the treated area of the skin, feet, and toes. The sodium lauryl sulfate provides an anionic surfactant to the lotion.

In another alternative embodiment, a “high potency formulation” with high concentrations of the active ingredients can be applied with physician monitoring for a few weeks, and then a “low potency formulation” can be applied as preventive medicine after the disease is in control. Thus, different concentrations for different stages of the disorder levels of the patient's skin may be formulated for the same simple treatment.

Patients received fast results, and reduction of the scales itching, and redness of the skin.

In alternative embodiments, the concentration of the anti-fungal and the anti-bacterial agents may be increased for feet treatment.

In other embodiments, pediatric patients formulae may be developed with low concentrations of the active ingredients.

In other alternative embodiments of this invention, the corticosteroid of the preferred embodiment may be substituted with nonsteroidal anti-inflammatory agents like the ones disclosed in U.S. Pat. No. 6,323,199 to Lehmann et al. Nevertheless, tests have not been conducted with the new anti-inflammatory agents to prove the success of the treatment if formulated with the new agents.

A method for treating psoriasis and other skin disorders and the symptoms associated therewith using the topical composition of this invention comprises topically applying an effective amount of the composition of this invention to the desired area twice per day. The composition is applied topically directly to the psoriatic lesions. The entire lesion should be covered with a thin layer of the composition. Testing to date has not shown any adverse effects from the composition to the native skin surrounding the lesion. However, it is recommended that the application of the composition be limited to the lesion and its periphery. The composition is allowed to dry on the patient's skin and is left in place on the patient's skin for a prescribed period, which is preferably from approximately one hour to 48 hours. The applied composition may be left uncovered, or it may be covered with a loose cloth bandage for cosmetic reasons. The composition is repeatedly applied in this manner until the desired results are obtained. Longer courses of treatment may be needed for more severe cases of psoriasis. Typically, the patient's skin will begin to show some signs of improvement when the first application of the composition is washed off of the skin and, after the course of the treatment, the plaque-like lesions will be replaced by healthy, normal skin. This method of treatment has been found to be effective on both new cases of psoriasis and on long-standing cases of psoriasis, which have not responded to other methods of treatment. Longterm follow-up of patients has shown relatively complete remission of the disease and restoration of normal skin growth for extended periods without recurring symptoms. The treatment has also been found to be free of undesirable systemic effects, tolerance buildup, and side effects which can result from known treatments.

A number of clinical trials have been done on human volunteers to demonstrate the efficacy of this treatment. The following are given as examples of the efficacy of treatment for severe, intractable cases of psoriasis, as well as new cases of psoriasis.

Example A—A 62 year old female patient with large, scaling patches of psoriatic lesions over 40% of her body underwent treatment using the composition and method of the present invention. After six weeks of treatment, the patient had experienced 100% clearance of the psoriatic lesions.

Example B—A 13 year old female patient with scaling patches of psoriatic lesions in almost 100% of her body underwent treatment using the composition and treatment of the present invention. After four weeks of treatment, the patient had experienced 75% clearance of the psoriatic lesions.

Example C—A 28 year old male with scaling patches of psoriatic lesions on his back and trunk underwent treatment using the composition and treatment of the present invention. After six weeks of treatment, the patient had experienced 90% clearance of the psoriatic lesions.

Example D—A 36 year old male with scaling patches of psoriatic lesions on his trunk, chest, and extremities underwent treatment using the composition and treatment of the present invention. After four weeks of treatment, the patient had experience 80% clearance of the psoriatic lesions.

Example E—A 49 year old male with scaling patches of psoriatic lesions on his back, trunk, and legs underwent treatment using the composition and treatment of the present invention. After four weeks of treatment, the patient had experienced 95% clearance of the psoriatic lesions.

Example F—A 45 to 50 year old male with psoriatic lesions covering almost 100% of his body underwent treatment using the composition and method of the present invention. After 6 weeks the patient had experience 85% clearance of the psoriatic lesions.

It is expected that total clearance is achieved in all cases after if treatment is continued for a longer period of time.

Clinical trials such as these have shown that the composition and method of treatment of the present invention are effective to provide rapid relief from symptoms in both new cases of psoriasis and longstanding cases of psoriasis which have not responded to other methods of treatment. In addition, longterm follow-up of patients has shown relatively complete remission of the disease and restoration of normal skin growth for extended periods without recurring symptoms.

All of the patents, patent applications, and publications recited herein, and in the Declaration attached hereto, if any, are hereby incorporated by reference as if set forth in their entirety herein. The components disclosed in the various patents, patent applications, and publications, disclosed or incorporated by reference herein may be used in the embodiments of the present invention, as well as equivalents thereof.

All, or substantially all, of the components and methods of the various embodiments may be used with at least one embodiment or all of the embodiments, if more than one embodiment is described herein.

The details in the patents, patent applications, and publications may be considered to be incorporable at applicant's option, into the claims during prosecution as further limitations in the claims to patentably distinguish any amended claims from any applied prior art.

Thus, there has been shown and described a topical composition for the treatment of psoriasis and other skin disorders and a method for its use which fulfills all the objects and advantages sought therefor. Although only a few exemplary embodiments of this invention have been described in detail above, those skilled in the art will readily appreciate that many modifications are possible in the exemplary embodiments without materially departing from the novel teachings and advantages of this invention. For instance, a different anti-oxidant or medication carrier may be used or the composition of the present invention may also find application for treatment of other skin conditions as well as other ploliferative diseases in other tissues of the body, other than those described above. Accordingly, all such modifications are intended to be included within the scope of this invention as defined in the following claims and their legal equivalents. In the claims, means-plus-function clauses, if any, are intended to cover the structures described herein as performing the recited function and not only structural equivalents but also equivalent structures.

Nothing in this application is intended to limit the composition of this invention from being mixed with any suitable substance that may facilitate administration, uniformity of distribution, enhance absorption, increase efficacy, or with any other substances that serve any other beneficial purpose, including adding substances currently in use for topical treatment of psoriasis that function by alternative mechanisms of action or complementary mechanisms of action, previously described under prior art treatments section of this application, or any new such drugs to come onto the market in the future.

In addition, nothing in this application is intended to limit additional compositions that may be added to or used in conjunction with composition of present invention. Although therapeutics of present invention may be topically administered in any suitable solution, any other suitable agent, carrier or delivery vehicle may be used. A wide variety of compositions including antibiotics, antibiotic creams, or non-toxic pharmaceutical carriers or vehicles, or the like, and in any suitable form such as a liquid, solid, semi-solid, ointment, lotion, paste, or the like may also be used where advantageous.

Nothing in the application is intended to limit the devices and methods used to facilitate application of composition of present invention. Devices and methods currently existing or to be developed in the future could be employed.

What is claimed is:

1. A composition for the topical treatment of psoriasis and other skin disorders, comprising:
(a) from about 0.01 to about 5 weight percent zinc pyrithione;
(b) from about 0.01 to about 5 weight percent triamcinolone acetonide;
(c) from about 0.01 to about 10 weight percent polysorbate 80;
(d) from about 5 to about 45 weight percent isopropyl myristate;
(e) from about 5 to about 45 weight percent ethyl alcohol;
(f) from about 2 to about 30 weight percent purified water;
(g) from about 0.01 to about 10 weight percent sodium lauryl sulfate;
(h) from about 0.01 to about 5 weight percent vitamin E;
(i) from about 0.01 to about 5 weight percent aloe vera;
(j) from about 0.01 to about 5 weight percent vitamin D3; and
(k) from about 0.01 to about 3 weight percent undercylenic acid.

2. A composition for the topical treatment of psoriasis and other skin disorders, comprising:
(a) from about 0.01 to about 5.0 weight percent Zinc Pyrithione;
(b) from about 0.01 to about 5.0 weight percent of a corticosteroid;
(c) from about 0.01 to about 10 weight percent Polysorbate 80;
(d) from about 0.01 to about 5.0 weight percent of an anti-fungal or anti-bacterial agent; and
(e) from about 0.01 to about 45% weight percent of at least one medication carrier.

3. The composition of claim 2, wherein said corticosteroid is triamcinolone acetonide.

4. The composition of claim 3, wherein said medication carrier is isopropyl myristate.

5. The composition of claim 3, wherein said medication carrier is selected from a group consisting of isopropyl myristate, ethyl alcohol, purified water, and sodium lauryl sulfate.

6. The composition of claim 4 or 5, further comprising at least one anti-oxidant selected from the group consisting of vitamin E, aloe vera, and vitamin D3.

7. The composition of claim 6, wherein the antifungal or antibacterial agent is undercylenic acid.

8. The composition of claim 7, wherein ethyl alcohol is from about 5 to about 45 weight percent.

9. The composition of claim 7, wherein purified water is from about 2 to about 30 weight percent.

10. The composition of claim 6, wherein sodium lauryl sulfate is from about 0.01 to about 10 weight percent.

11. The composition of claim 5 wherein said carrier is in the form of a member selected from the group consisting of a lotion, spray, ointment, cream, gel, emulsion and shampoo.

12. A method for treating psoriasis and other skin disorders and the symptoms associated therewith comprising:
topically applying an effective amount of the composition of claim 9 or 10 to an area desired at least twice per day.

13. A method for topical treatment of psoriasis comprising applying to a psoriatic lesion a composition comprising zinc pyrithion, polysorbate 80, triamcinolone acetonide, and isopropyl myristate.

14. The method for topical treatment of psoriasis of claim 13, further comprising repeatedly applying said composition to the psoriatic lesion at intervals until the psoriatic lesion goes into remission.

15. The method for topical treatment of psoriasis of claim 14, further comprising repeatedly applying said composition to the psoriatic lesion, and allowing said composition to remain on the psoriatic lesion for a period of approximately three to six weeks.
